(19) United States
(12) Patent Application Publication
(10) Pub. No.: US 2025/0134801 A1
(43) Pub. Date: May 1, 2025

(54) NOVEL METHODS FOR DECELLULARIZING EXTRACELLULAR MATRIX

(71) Applicant: RegenX Science, Inc., West Palm Beach, FL (US)

(72) Inventors: Ramon B. Montero, Boca Raton, FL (US); Jun-Hung Cho, Boca Raton, FL (US); Elisabeth A. Corson, West Palm Beach, FL (US)

(73) Assignee: RegenX Science, Inc., West Palm Beach, FL (US)

(21) Appl. No.: 18/837,137

(22) PCT Filed: Feb. 10, 2023
(86) PCT No.: PCT/US2023/062398

§ 371 Date: Aug. 8, 2024

Related U.S. Application Data
(60) Provisional application No. 63/267,863, filed on Feb. 11, 2022.

(51) Int. Cl. A61K 8/98; A61K 35/65; A61K 45/06; A61Q 17/04; A61Q 19/08

(52) U.S. Cl. CPC A61K 8/987; A61K 35/65; A61K 45/06; A61Q 17/04; A61Q 19/08; A61K 2800/805

(57) ABSTRACT

The present disclosure describes a novel method for decellularizing amphibian extracellular matrix to obtain an isolated ECM that is substantially free of cellular and nuclear debris. The method includes washing the amphibian ECM in solutions containing one or more surfactants, incubating the ECM with one or more proteases, and incubating the ECM with one or more nucleases. The present disclosure also describes various methods of using the isolated ECM.

### CROSS-REFERENCE TO RELATED APPLICATIONS

This application claims the benefit of U.S. Provisional Application No. 63/267,863 filed Feb. 11, 2022, which is incorporated herein by reference in its entirety.

### TECHNICAL FIELD

The disclosure describes a novel method for decellularizing the extracellular matrix and compositions comprising the decellularized extracellular matrix for various uses.

### BACKGROUND

The skin, the largest organ of the mammalian body, is an outer covering of the body which serves three main functions: protection against microbes and excessive water loss, regulation of body temperature, and sensation of touch, heat, and cold. Mammalian skin has two primary layers, the epidermis, and the dermis. The epidermis, the outermost layer of the skin which prevents microbes from entering and keeps water in our body, is a stratified squamous epithelium composed of keratinocytes. The dermis is the layer of skin beneath the epidermis which serves as a location for the appendages of the skin and provides elasticity to the skin through an extracellular matrix composed of collagen fibers, elastic fibers, hyaluronan, and proteoglycans. The dermis and the epidermis are separated by a thin sheet of fibers called the basement membrane which regulates the flow of cells and molecules, such as cytokines and growth factors, between the dermis and epidermis, during the remodeling, repair, and regeneration process. Underneath the dermis is the hypodermis which is composed of loose connective tissue, such as fat, and elastin. The primary cells of the hypodermis include fibroblasts, macrophages, and adipocytes.

An injury or a disease creates an interruption of the morphology and function of an organ or tissue, such as the skin. The remodeling and repair of the organ or tissue following an injury is a complex wound healing process involving interactions between cells, growth factors, and extracellular matrix (ECM). The process in adult mammals involves well-known stages: homeostasis, inflammation, proliferation, maturation, and remodeling. During homeostasis, clotting takes place to stop the bleeding. Inflammation involves the recruitment of white blood cells, antibodies, nutrients, and enzymes to the affected area to accelerate wound healing. During proliferation, new healthy granulation tissues, such as new connective tissues and blood vessels, replace the wound. Maturation and remodeling take place after the wound is closed and involve the repair of the dermal tissues to improve their tensile strength.

In contrast to the repair process in which the goal is to re-establish function without regard to the exact placement of injured tissue, regeneration is the replacement of injured tissue with an exact copy such that both morphology and functionality are completely restored. As an example, non-injured skin undergoes complete regeneration continually with the replacement of new cells. However, injured adult mammalian skin does not regenerate completely and heals with a scar.

In contrast to mammals, Urodele regenerates its skin structures including the dermis and secretion glands without forming a scar after a deep skin injury. It has been reported that the extracellular matrix (ECM) of Urodeles may possess desirable characteristics important to wound healing, making them an ideal source of biomaterial for wound healing, xenotransplantation, and other skin conditions. Accordingly, the ECM of Urodele can provide models for tissue repair and regeneration in mammals and starting materials for compositions for treating mammalian tissues including the skin.

The ECM of Urodele contains native cells and genetic materials for maintaining structural and biochemical functions and properties. However, the native cells and genetic materials of the Urodele ECM can induce undesirable effects and immune responses in a subject. Decellularization is a process in which native cells and genetic materials are removed from the tissue without affecting the structural and functional integrity of the tissue. The decellularized tissue can then be used in a subject. However, there is a need to develop an improved method of obtaining decellularized Urodele ECM that can be used effectively in subjects, especially mammalian subjects.

### SUMMARY

This Summary is not intended to identify key features or essential features of the claimed subject matter, nor is it intended to be used to limit the scope of the claimed subject matter.

The present disclosure provides new and improved methods of decellularizing a biological sample of an amphibian. In embodiments, the biological sample contains ECM and is harvested from a neotenic Urodele (for example, the Ambystoma hybrid species) or a larval stage young Apoda (frog/tadpole) species.

The method includes washing the sample with one or more surfactants and incubating the sample with one or more proteases and one or more nucleases. In embodiments, the method includes washing the sample with one or more detergent solutions, treating the sample with one or more proteases, and treating the protease treated sample with one or more nucleases to obtain an isolated ECM that can be used in vitro and/or in vivo. The one or more detergent solutions include one or more non-ionic surfactants, such as Triton™ X-100 Reduced, and/or an anionic detergent, such as N-lauryl sarcosine (NLS). The one or more proteases include an amino-endopeptidase, such as Dispase II. The one or more nucleases include a DNA/RNA endonuclease, such as Benzonase® nuclease. After decellularization, the isolated ECM sample can be micronized and dehydrated for storage.

The present disclosure also describes isolated ECM and compositions comprising isolated ECM. The isolated ECM has greater than 70% of its cells removed yet the structural and/or functional architecture of the ECM remains intact. The isolated ECM includes less than 20 ng/mg of dry ECM (or less than 10 ng/μl of DNA extract). The length of residual DNA fragments in the isolated is less than 300 base pairs (bps).

The isolated ECM and compositions can be used for organ and tissue regeneration, as implants, and for treating skin conditions.

### BRIEF DESCRIPTION OF THE DRAWINGS

FIGS. 1A, 1B, 1C, 1D, 1E, and 1F show slides of the isolated ECM stained with hematoxylin and eosin and imaged via light microscopy. FIGS. 1A-1D and 1B show samples washed with Triton X-100. FIGS. 1E and 1F show samples washed with NLS.

FIGS. 2A and 2B show the amount of DNA in the isolated ECM. FIG. 2A shows average DNA quantification per sample. FIG. 2B shows the percentage of DNA converted from the concentration shown in FIG. 2A.

FIGS. 3A and 3B show 1% agarose gels with a DNA ladder. FIG. 3A shows residual DNA from Naïve tissue and isolated ECM samples (Triton X-100 (T1 or T2) or NLS samples (NLS) from the bottom left corner (BLC) or center (C) of isolated ECM sample: T1_BLC, T_C, T2_BLC, T2_C, NLS_BLC, and NLS_C). FIG. 3B shows residual DNA from isolated ECM samples in triplicates (3×, three lanes).

FIGS. 4A and 4B show average particulate length with error bars for the cryomilled samples. The average length for T1 and NLS is 28.36 μm±13.19 and 30.05 μm±34.70, respectively. The average area for T1 and NLS of 494.06 μm2±293.2 and 631.5 μm2±550.91, respectively.

### DETAILED DESCRIPTION

The terms “a,” “an,” “the” and similar referents used in the context of describing the invention (especially in the context of the claims) are to be construed to cover both the singular and the plural, unless otherwise indicated herein or clearly contradicted by context.

The term “antigen” refers to a molecule that is a toxin or is foreign to the subject and induces an immune response in the form of the production of antibodies against the molecule. The isolated ECM described herein can have reduced antigenicity as compared to a native ECM, such that it can be used in a subject.

The term “bioactivity” refers to biological effects. A substance is bioactive or has bioactivity includes a substance having a biological function.

The term “biocompatible” refers to a product and its normal degradation products in vitro, ex vivo, or in vivo that are substantially non-toxic and non-carcinogenic to a cell, tissue, organ, organism, or subject within useful, practical, and/or acceptable tolerances. The term “cytocompatible” refers to a product that can sustain the viability and growth of a population of cells.

The term “biomaterial” refers to a material suitable for in vitro, ex vivo, or in vivo use. As an example of in vivo use, the biomaterial is suitable for administering to a subject in need thereof. The material can be synthetic or natural. The decellularized ECM (isolated ECM) described herein is an example of a biomaterial.

The term “carrier” or “excipient” refers to a substance added to a composition that does not affect the active compound in the composition. The carrier can be a diluent. The excipient can be a substance added to the composition to facilitate the administration of the composition.

The term “cosmetics” refers to products (excluding pure soap) intended to be applied to the human body for cleansing, beautifying, promoting attractiveness, or altering the appearance. Examples of cosmetic benefits can include improving the appearance of skin such as improving the appearance of wrinkling and fine lines, removing oil and excess sebum, reducing the appearance of skin blemishes, cleansing, and conditioning the skin, toning, and tightening the skin, soothing irritation, and refreshing and cooling the skin.

The term “drugs,” “pharmaceuticals,” or “therapeutics” refers to articles intended for use in the diagnosis, cure, mitigation, treatment, or prevention of disease and articles (other than food) intended to affect the structure or any function of the body of man or other animals.

The term “derive”, “derived,” or “derives” refers to a product obtained from any stated source by any useful method. For example, an extracellular matrix (ECM) derived from an amphibian refers to an ECM obtained from a member of the amphibian family.

The term “exogenous” refers to a product that originated outside of the organism, tissue, cell, organ, or subject. In contrast, the term “endogenous” refers to a product that originated from the organism, cell, tissue, organ, or subject.

The term “extracellular matrix” or “ECM” refers to a natural scaffolding having a three-dimensional structure including biomolecules and minerals that provide biochemical support to surrounding cells. The ECM includes structural and non-structural biomolecules, such as collagens, elastins, laminins, glycosaminoglycans, proteoglycans, antimicrobials, chemoattractants, cytokines, and/or growth factors. The ECM can be obtained from various sources of tissues including the skin and non-cutaneous tissues.

The term “decellularized extracellular matrix” or “decellularized ECM” refers to ECM prepared by removing and/or devitalizing cells from ECM found in multicellular organisms, for example, amphibians or mammals. Decellularized ECM is substantially free of intact cells, lysed cells, and cellular components including cellular and nuclear debris such that the decellularized ECM exhibits reduced immunogenicity so that it can be administered to subject, for example, a mammalian subject, as a non-toxic xenograft or biomaterial. A decellularized ECM that is “substantially free of immunogenic components” refers to an ECM in which immunogenic components are at a level that is not sufficient to induce an adverse immune response in a subject.

The term “isolated” refers to being separated or removed from its native surroundings, such that it is substantially free from components that accompany it in its naturally-occurring state. For example, a cell or a protein can be isolated from its naturally-occurring state. The term “isolated ECM” refers to decellularized ECM.

The term “immunogenic” refers to relating to or producing an immune response. The term “immunogenicity” refers to the ability of a foreign substance, such as an antigen, to provoke an immune response in a subject. The isolated ECM described herein can have reduced immunogenicity as compared to the native ECM, such that it can be used as a biomaterial in a subject.

The term “native ECM” or “naïve ECM” refers to a naturally occurring amphibian ECM (or corresponding ECM sample) that has not been decellularized.

The term “non-toxic” refers to a product that causes little or no adverse reaction or substantial harm to cells and tissues in vitro or ex vivo, and/or does not cause substantial adverse or undesirable reaction or substantial harm to cells and tissues in the body (in vivo).

The terms “prevent” or “prevention” refers to the prevention of the onset, recurrence, or spread of a condition or one or more symptoms of the condition. As an example, the condition could be a skin condition. The term includes the administration of a product described herein before the onset of symptoms in particular to subjects at risk of developing a condition, such as a skin condition. The term includes the inhibition or reduction of one or more symptoms associated with the skin condition. The term “prevention” can be used interchangeably with the term “prophylactic treatment”.

The term “retain structural and functional integrity” used with reference to the ECM refers to retaining sufficient structure and function to permit and support the use of the matrix as a substrate for the growth of cells in vivo, ex vivo, or in vitro. For example, the isolated ECM retains the structure and functional properties of a naturally occurring ECM enabling its use as a biomaterial.

The terms “scaffold” and “bioscaffold” are used interchangeably to refer to a substrate on which cells can grow in vitro, ex vivo, and/or in vivo. A scaffold or bioscaffold is an example of a biomaterial.

The term “skin conditions” includes skin conditions that require therapeutic “drug” treatment including diseases, defects, and injuries including wounds.

The term “cosmetic skin conditions” includes skin conditions that are related to tone, clarity, radiance, brightness, and/or hydration of the skin.

The term “subject” refers to an animal, for example, a mammal. Examples of mammals include a human, a dog, a cat, a horse, a cow, a goat, a sheep, a pig, or a non-human primate. A subject in need of treatment or a subject in need thereof includes a subject having a disease or condition that needs to be treated. A subject in need thereof also includes a subject that needs treatment and/or prevention of a skin condition.

The term “therapeutically effective amount” refers to an amount of a product or composition that provides a therapeutic benefit in the treatment, prevention, or management of a condition or disease, such as a skin disease, an injury to the skin, or a wound, for example, a drug product. The term “therapeutically effective amount” also includes that amount of a compound that, when administered, is sufficient to prevent the development of, or alleviate to some extent, one or more of the signs or symptoms of the disorder or disease being treated.

The term “treatment” or “treating” in the context of administering a product, such as a biomaterial, to a subject refers to administering the product to achieve a desirable clinical/medical end-point, including alleviating symptoms of a disease or condition. Examples of such desirable end-points associated with skin disease or condition include wound healing, tissue closure, bulking tissue, preventing tissue adhesion, providing structural support to tissue, providing a protective barrier, and/or correcting a defect. Administering the product also includes applying the product on a subject.

The term “xenogenic” refers to a product derived or originated from a member of another species.

The term “amphibians” refers to cold-blooded vertebrate animals that include frogs, toads, newts, and salamanders. They have an aquatic gill-breathing larval stage followed by a terrestrial lung-breathing adult stage. The amphibians described herein are young or neotenic amphibians. A young amphibian includes a young frog, such as a froglet, tadpole, or larval stage young Apoda.

The term “salamanders” refers to a group of amphibians characterized by a lizard-like appearance and having a tail throughout life. There are ten families of salamanders, and they include the Ambystomatidae (mole salamanders), Amphiumidae (Congo eels), Cryptobranchidae (giant salamanders), Dicamptodontidae (Pacific giant salamanders), Hynobiidae (Asiatic salamanders), Plethodontidae (lungless salamanders), Proteidae (mudpuppies and olms), Rhyacotritonidae (torrent salamanders), Salamandridae (newts and true salamanders), and Sirenidae (sirens). The ten families of salamanders are grouped together under the order Urodela (or Caudata). The term “Urodele” refers to a salamander of the order Urodela, in the class Amphibia. The salamanders can be from the orders Urodela and Apoda.

Urodeles begin life as aquatic animals in a larval state, and some undergo metamorphosis from a juvenile form with gills to an adult, terrestrial, air-breathing form with lungs. During metamorphosis, a Urodele's physical features are altered in preparation for life on land. These alterations include caudal fin resorption, thickening of the skin, the development of dermal glands, and resorption of gills. Sexual maturity also occurs during this time in most Urodeles. However, some families of Urodeles are “neotenic,” which means that individuals of such families, even after reaching sexual maturity, retain their juvenile aquatic form throughout their lives. The axolotl (Mexican walking fish), Ambystoma mexicanum, and/or hybrids of A. mexicana and A. tigrinum are examples of neotenic salamanders. Instead of becoming a terrestrial amphibian, an adult axolotl remains aquatic and gilled. However, under certain circumstances, an axolotl will undergo metamorphosis and transform into a terrestrial form.

Axolotls have the ability to fully regenerate lost or damaged body parts including organs, limbs, and parts of the central nervous system, throughout their entire life. Aquatic axolotls undergo rapid re-epithelialization during wound healing and limb regeneration, both of which are scar-less processes. Similarly, metamorphic terrestrial axolotls, which retain several larval skin features, also exhibit scar-free wound healing, but at a slower rate than their aquatic, pre-metamorphic counterpart. The axolotl wound healing process resembles the scar-free healing process of mammalian fetal and embryonic wounds. Such wounds exhibit re-epithelialization and basement membrane reformation that occur at a faster rate than do the corresponding events in postnatal mammals. Moreover, similar to the human amniotic membrane (AM), the axolotl ECM is rich in growth factors, which are favorable for wound healing.

The ECM is a three-dimensional network of extracellular macromolecules and minerals including collagen, enzymes, glycoproteins, and hydroxyapatite which provide structural and biochemical support to surrounding cells. The ECM can include a combination of fibrous and network type collagens. Examples of various types of collagens, such as one or more of type I, II, III, IV, V, and VI collagens. The ECM can also include elastin and/or elastic fibers. The ECM can also include laminin, fibronectin, hyaluronan, chondroitin sulfate, or both, and/or one or more proteoglycan, glycoprotein, glycosaminoglycan, or any combination thereof. The components and structure of the ECM play an important role in the healing process because the ECM components create scaffolding which provides the structural architecture of the matrix required for the healing process. Moreover, the ECM components are involved in stimulating the adhesion and migration of cells during the healing process as well as mediating the interactions among the cells and between the cells and the matrix, or between ECM proteins during the healing process. Further, the ECM components also serve as a reservoir and modulator of the action of the cytokines and growth factors to regulate wound repair activities. However, the ECM contains cells and genetic materials that need to be removed before it can be used as a biomaterial since the cells and genetic material can induce an adverse immune response.

Decellularization of the ECM is the removal of cells and cellular components from the ECM of a biological sample, such as a tissue, while retaining the ECM proteins and the native ECM architecture or structure for effective use as a biomaterial. The present disclosure describes a novel method of decellularizing amphibian ECM, for example, Urodele ECM, for use in preventing and/or treating and healing various conditions including skin conditions. In contrast to known methods of decellularizing ECM which can involve the use of cross-linking agents and other harsh agents, the method described herein uses milder agents yet provides a decellularized amphibian ECM containing fewer cells and cellular debris, thus enabling the decellularized ECM to be used in vitro, ex vivo, and/or in vivo without causing undesirable adverse effects.

The method of decellularizing amphibian ECM includes harvesting or obtaining an amphibian sample, washing the sample, and incubating the sample with one or more proteases and one or more nucleases. In embodiments, the method of decellularizing amphibian ECM includes harvesting an amphibian sample, washing the sample, incubating the sample with one or more proteases, washing the protease treated sample, incubating the washed protease treated sample with one or more nucleases to obtain an isolated ECM that can be used as a biomaterial in vitro, ex vivo, and/or in vivo.

The amphibian sample includes any biological sample from an amphibian, especially a young amphibian, such as a young frog or neotenic Urodele, that includes an ECM and can be decellularized and used. The biological sample can include any amphibian tissue such as connective tissues, adipose tissue, bone, blood plasma, skin, cartilage, tendon, dura mater, and fascia. Other tissues include the dermis, basement membrane, and epithelial tissue, for example, basement membrane or epithelial tissues that line the body cavities such as the parietal mesothelial tissues of the thoracic cavity, the abdominal cavity, and the pericardium. In embodiments, the biological sample is tissue from a young amphibian, such as a young frog, or neotenic Urodele. The biological sample can be a fresh sample or a frozen sample that has been thawed. The sample can be obtained from any Urodele species including axolotl. In embodiments, the sample is obtained from the skin of an axolotl.

An amphibian sample is obtained or harvested from a young amphibian, such as a neotenic Urodele or a young frog. A sample is surgically removed from a tissue of a young amphibian. The sample can be a full-thickness explant of various shapes and sizes. The sample can be 5 by 5 centimeters (cm) in size or larger. The sample is cleaned and prepared for decellularization.

The method of decellularization described herein includes briefly rinsing the sample with one or more solutions including water or buffered solution and washing with a detergent solution with agitation prior to protease treatment. Examples of buffered solutions include phosphate-buffered saline (PBS), Dulbecco's PBS (DPBS), tris-buffered saline (TBS), and HEPES (4-(2-hydroxyethyl)-1-piperazineethanesulfonic acid). Detergents include non-denaturing non-ionic and anionic surfactants that are mild on the ECM and easy to use, for example, can be used at room temperature without raising or lowering the temperature or pressure. It was found that non-ionic surfactants such as Triton™ X-100 (2-[4-(2,4,4-trimethylpentan-2-yl)phenoxy]ethanol) and Triton™ X-100 Reduced (Polyoxyethylene (10) isooctylcyclohexyl ether) and secondary alcohol ethoxylates such as TERGITOL™ (secondary alcohol ethoxylate) such as TERGITOL™ 15-2-9 (polyglycol ether surfactant) and TERGITOL™ TMN-10 (trimethylnonylpolyethylene glycol, polyethylene glycol trimethylnonyl ether) and anionic detergents such as N-lauryl sarcosine (NLS), potassium lauryl sarcosine, cholate, deoxycholate, sodium dodecyl sulfate (SDS) are useful mild detergents for the decellularization amphibian biological samples. Other non-ionic surfactants include polyoxyethylene sorbitan fatty acid ester or Tween such as Tween 20, 40, 60, or 80.

The sample can be first rinsed with water or buffered solutions for 3 to 10 minutes (mins) at room temperature, and subsequently washed with detergents at 37° C. with agitation. Exemplary wash conditions for detergents include the following: a 5 by 5 cm amphibian sample is washed twice with 20 to 30 mL of detergent in buffer, such as DPBS, at a concentration of 0.1% to 1.0% v/v for 3 to 12 hours (hrs), each time at 37° C. with agitation at 90-120 rotations per minute (RPM). In embodiments, the sample is washed briefly for 5 mins with DPBS at room temperature, followed by washing with Triton, for example, Triton X-100 or Triton X-100 Reduced, or with NLS for 6 hrs at 37° C. with agitation at 100 RPM, and subsequently rinsed with buffered solution, such as PBS or DPBS, for 5 mins at room temperature before being treated with one or more proteases.

The sample can also be washed with a non-detergent solution containing latracunlins (Lats) or cytochalasins, instead of a detergent solution. The Lats are cytochalasins which are actin depolymerization agents. They bind actin monomers near the nucleotide-binding cleft and prevent actin monomers from polymerizing. Examples of Lats include Lat A and Lat B. Examples of cytochalasins include cytochalasin A, B, C, D, E, F, H, and J. The Lats have a nanomolar range potency. An exemplary non-detergent process using Lats for a 5 by 5 cm tissue sample would be as follows: incubate the sample in 100 ml of 50 nanomolar (nM) Lat, for example, Lat B, in high glucose DMEM for 2 hrs at 37° C., wash with 100 ml double distilled water for 15 mins, incubate with 100 ml 0.6 M potassium chloride for 90 mins, incubate in 100 mL 1.0 molar (M) potassium iodide for 90 mins, wash with 200 ml distilled water overnight, incubate with potassium chloride, incubate with potassium iodine, and wash with distilled water overnight. The washes and incubation with high ionic solutions are performed at room temperature.

The washed amphibian sample is subsequently incubated with one or more proteases for about one to two hours at 37° C. to remove the epidermis and disrupt cell attachment to the dermal matrix. The concentration of the protease is at least 1 unit of enzyme per ml of solution or 2 to 10 units/mL, 2 to 7 units/mL, 2 to 5 units/mL, or 2 to 4 units/mL of solution. The one or more proteases for treating the amphibian sample include amino-endopeptidases, which are relatively gentle, dissociate well at physiological temperature and pH, and are able to maintain the cell membrane integrity which makes the method easier to perform. Examples of amino-endopeptidases include dispase such as dispase I and dispase II, trypsin, papain, and collagenase. In embodiments, the protease for treating the sample is dispase II. It was found that dispase Il has a mild proteolytic action which helps in maintaining cell membrane integrity and is easy to use as it can be used at physiological temperature and pH. In embodiments, a 5 by 5 cm tissue sample is incubated with 2 units of dispase II per mL of DPBS for 90 mins at 37° C. In embodiments, the dispase is dispase II (CAS #42613-33-2).

The protease treated sample is then rinsed briefly with one or more solutions including water or buffered solution at room temperature. In embodiments, the protease treated sample is rinsed with buffered saline, such as PBS or DPBS, for 5 mins.

After briefly rinsing with PBS or DPBS, the protease treated sample is then incubated with one or more nucleases at 37° C. for 3 to 12 hrs with agitation at 90 to 120 RPM. The concentration of the nuclease is at least 30 units/ml of solution, or 40 to 100 units/ml, 40 to 75 units/ml, 40 to 60 units/ml, or 40 to 50 units/ml of solution. In this step, a nonspecific DNA/RNA endonuclease is used to remove nucleic acids from the sample. Examples of nonspecific endonucleases include benzonuclease and turbonuclease. Other nucleases include recombinant DNA nuclease, pulmozyme, and a combination of DNases and RNases. In embodiments, the protease treated sample after washing is treated with 40 units/ml benzonuclease for 6 hrs. After nuclease treatment, the sample is briefly rinsed with buffered solution, such as PBS or DPBS at 37° C., and subsequently washed with buffered solution, such as PBS or DPBS at 37° C. for 24 hours with shaking at 100 RPM. In embodiments, the nuclease is benzonase (CAS #9025-65-4).

In embodiments, the sample is washed with Triton X-100 Reduced, treated with dispase II, treated with benzonuclease, and washed with DPBS. In between each of these steps, the sample can be briefly rinsed for five minutes with buffered saline such as PBS or DPBS which uses three times the volume of the solution of each of the steps.

Optionally, the isolated ECM after the nuclease treatment and washing can be stored, dialyzed, disinfected, sterilized, micronized, dehydrated (lyophilized), and/or turned into other forms for suitable use. The isolated ECM can be stored at −20° C. after the nuclease treatment. The isolated ECM can also be preserved in a 10% formalin neutral buffer.

The isolated ECM can be sterilized by using gamma irradiation, electron-beam (e-beam), glutaraldehyde, ethylene oxide, peracetic acid (PAA), ethanol, or CO2 based technology. In embodiments, the isolated ECM is sterilized using CO2 based technology which can sterilize soft material without compromising the potency of the material. The isolated ECM can be micronized by sieving, cryomilling, mincing, spray drying, jet milling, or supercritical fluid (SCF) technology. The isolated ECM can also be lyophilized to remove water at a low temperature which can be achieved in a desiccator or a low-temperature vacuum oven.

The isolated ECM can be micronized to a particle size of about 1 to 1000 microns, 1 to 900 microns, 1 to 800 microns, 1 to 700 microns, 1 to 600 microns, 1 to 500 microns, 1 to 400 microns, 1 to 300 microns, 1 to 200 microns, 100 to 300 microns, 100 to 250 microns, 100 to 200 microns, 100 to 150 microns, or 100 to 125 microns. In embodiments, the size of the particles is about 100 to 500 microns. The micronized particles can be immediately dehydrated by lyophilization and stored at room temperature after lyophilization.

Lyophilization can include freezing under a vacuum and one or more cycles of drying at a low temperature under a vacuum. Examples of the temperatures for freezing the isolated ECM for lyophilization can include −25° C. to −60° C., −25° C. to −50° C., −25° C. to −45° C., −30° C. to −45° C., −35° C. to −45° C., or −45° C. Examples of the temperature for drying the isolated ECM sample for lyophilization can include 10° C. to −50° C., 10° C. to −45° C., 10° C. to −40° C., 5° C. to −40° C., 5° C. to −30° C., 5° C. to −25° C., 0° C. to −25° C., −40° C., −25° C., or 5° C. Examples of pressure for freezing and/or drying the isolated ECM sample during lyophilization include 25 mTorr to 375 mTorr, 25 mTorr to 360 mTorr, 50 mTorr to 360 mTorr, 75 mTorr to 360 mTorr, 100 mTorr to 360 mTorr, 150 mTorr, to 325 mTorr, 175 mTorr, to 300 mTorr, 200 mTorr to 300 mTorr, 225 mTorr to 275 mTorr, 360 mTorr, 300 mTorr, 250 mTorr, 100 mTorr, or 50 mTorr. The length of time for drying and/or freezing can range from 5 minutes to 800 minutes, 5 minutes to 750 minutes, 5 minutes to 720 minutes, 5 minutes to 400 minutes, 90 minutes to 350 minutes, 10 minutes to 720 minutes, 5 minutes, 120 minutes, 300 minutes, or 720 minutes. In embodiments, the isolated sample is lyophilized in the first stage by freezing for 5 minutes at 360 mTorr, followed by a first phase of drying at −40° C. for 120 minutes at 100 mTorr, −25° C. for 300 minutes at 50 mTorr, and −25° C. for 720 minutes at 250 mTorr, and followed by a second phase of drying at 5° C. for 120 minutes at 300 mTorr.

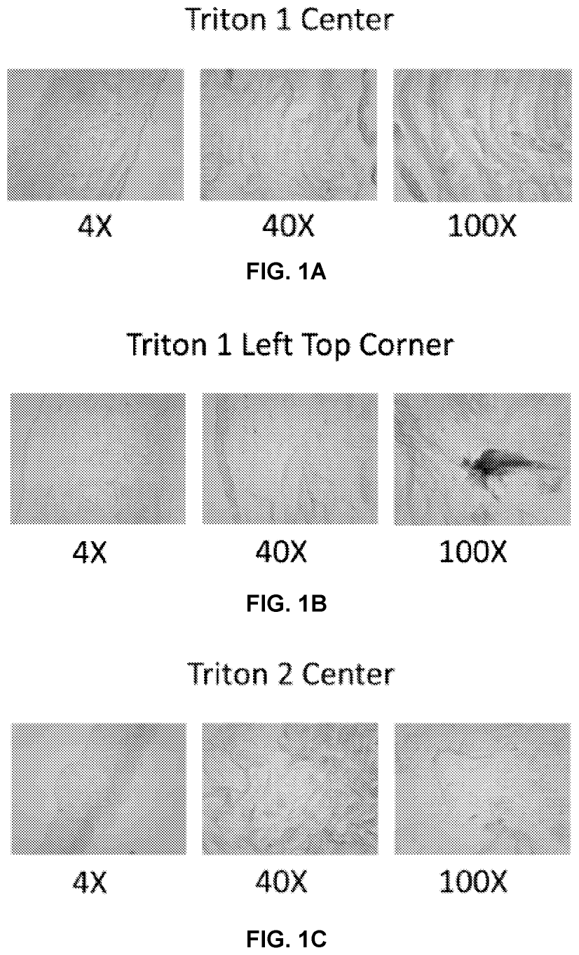

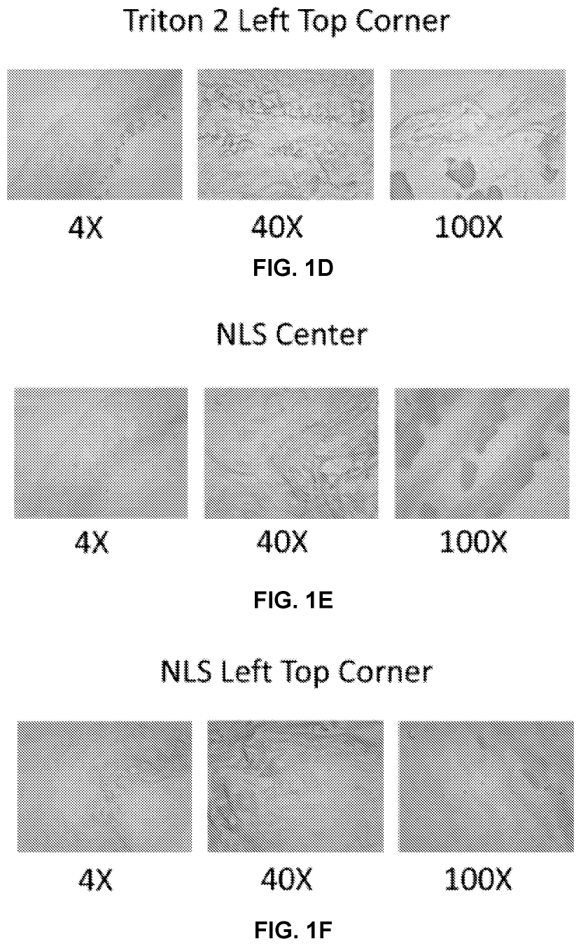

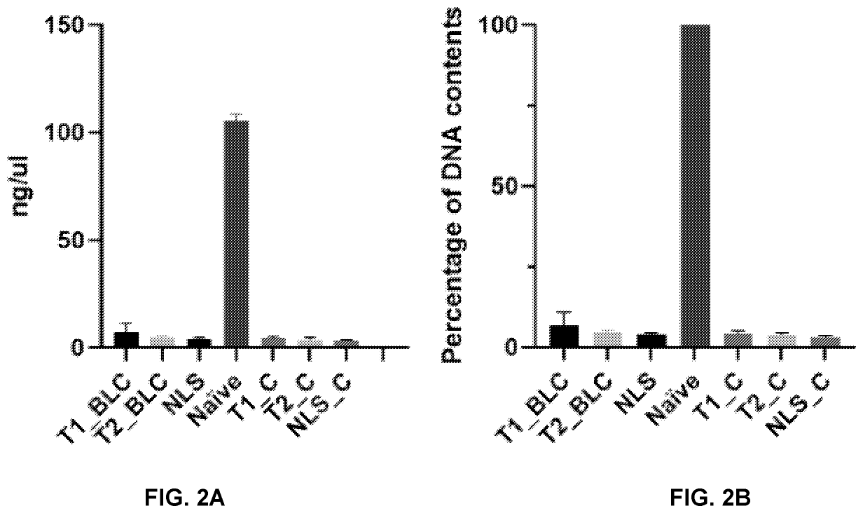

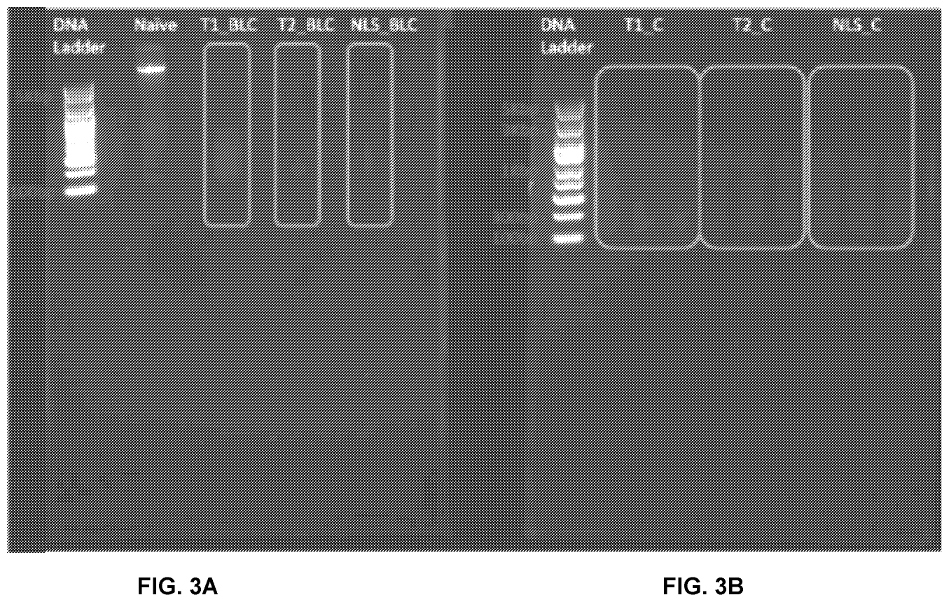

After decellularization, the isolated ECM can be analyzed by staining for visualization of the integrity of the structure of the decellularized ECM and the removal of cells by light microscopy, and quantitation of the amount of DNA. The staining methods include Hematoxylin and Eosin staining (H&E staining) and DAPI (4′6-diamidino-2-phenylindole). Hematoxylin is a basic dye that stains the acidic components of the cells, such as the nucleus, while Eosin is the acidic dye that stains the basic components of the cells, such as the cytoplasm. DAPI is a blue-fluorescent DNA stain. Surprisingly, it was found greater than 70% of the cells were removed from the isolated ECM, yet the ECM architecture remained intact (See FIGS. 1A-1F). Moreover, the novel method of decellularization described herein removed a significant amount of the DNA from the cells. The isolated ECM contains less than 10 ng of DNA, which is about less than 10% of the total DNA in the native (naïve) ECM (See FIGS. 2A-2B). Moreover, the length of the residual DNA fragments in the isolated ECM is less than 300 bps in length (See FIGS. 3A-3B), while the length of the DNA in the native ECM is greater than 250 kilobases (kbps) in length.

The present disclosure describes an isolated ECM having greater than 70% of its cells (native cells) removed, yet it has the structural and/or functional architecture of a native amphibian ECM. The isolated ECM has greater than 99%, 95% to 99%, 90% to 99%, 71% to 99%, 75% to 95%, 80% to 90%, or 80% to 85% of its native cells removed. The isolated ECM has greater than 75%, 80%, 85%, 86%, 87%, 88%, 89%, 90%, 91%, 92%, 93%, 94%, 95%, 96%, 97%, 98%, or 99% of its native cells removed. The isolated ECM includes less than 30% of its native cells. The isolated ECM has 1% to 29%, 5% to 25%, 10% to 20%, or 15% to 20% of its native cells removed. The isolated ECM contains less than 1%, 2%, 3%, 4%, 5%, 6%, 7%, 8%, 9%, 10%, 11%, 12%, 13%, 14%, 15%, 20%, or 25% of its native cells. The isolated ECM contains a non-detectable amount of its native cells.

The amount of residual DNA in the isolated ECM described herein is less than 1ng/ul (ng DNA per microliter of extract) or is only 1 to 10 ng/μl, 2 to 9 ng/μl, 3 to 8 ng/μl, 3 to 7 ng/μl, 3 to 6 ng/μl, 3 to 5 ng/μl, 3 to 4 ng/μl, 2 to 3 ng/μl, 4 to 5 ng/μl, 5 to 6 ng/μl, 6 to 7 ng/μl, 7 to 8 ng/μl, 2.5 ng/μl, 3 ng/μl, 3.5 ng/μl, 4 ng/μl, 4.5 ng/μl, 5 ng/μl, 5.5 ng/μl, 6 ng/μl, 6.5 ng/μl, 7 ng/μl, 7.5 ng/μl, or 8.0 ng/μl (ng DNA per microliter of extract). In contrast, the native (naïve) ECM contains DNA in the amount of 100 ng/μl (DNA per microliter of DNA extract solution). As compared to the native ECM, the amount of residual DNA in the isolated ECM is less than 1% of the amount of the DNA in the native ECM or is only 1% to 10%, 2% to 9%, 2% to 5%, 3% to 8%, 3% to 7%, 3% to 6%, 3% to 5%, 3% to 4%, 2% to 3%, 4% to 5%, 5% to 6%, 6% to 7%, 7% to 8%, 1%, 2%, 2.5%, 3%, 3.5%, 4%, 4.5%, 5%, 5.5%, 6%, 6.5%, 7%, 7.5%, or 8.0% of the amount of DNA in the native ECM. The amount of residual DNA in the isolated ECM is non-detectable.

The amount of DNA in isolated ECM described herein is less than 20 ng/mg of dry ECM. In embodiments the amount of DNA in the isolated DNA is 10 to 19 ng/mg, 10-18 ng/mg, 10 to 17 ng/mg, 10 to 16 ng/mg, 10 to 15 ng/mg, 19 ng/mg, 18 ng/mg, or 17 ng/mg of dry ECM.

Further, the residual DNA fragments in the isolated ECM are less than 300 bps in length (See FIG. 3). The residual DNA fragments in the isolated ECM are of non-detectable length, less than 50 bps, or between 50 to 300 bps, 60 to 290 bps, 70 to 280 bps, 80 to 270 bps, 90 to 260 bps, 50 to 250 bps, 100 to 250 bps, 100 to 240 bps, 100 to 230 bps, 100 to 220 bps, 100 to 210 bps, or 100 to 200 bps in length. In embodiments, the residual DNA fragments in the isolated ECM are less than 250 bps in length. Thus, the length of the residual DNA fragments in the isolated ECM is substantially small as compared to the native ECM which is greater than 250 kbps in length.

The decellularization process described herein provides an isolated ECM in which its interstitial structure is substantially intact and has bioactivity. The isolated ECM has sufficient structural and functional properties and/or reduced immunogenicity, such that it can be used as a biomaterial in vitro, ex vivo, and/or in vivo. The isolated ECM has a substantial native structure, such that cells can grow on it. The isolated ECM can be used as a scaffold for adhesion, proliferation, differentiation, regeneration of cells, and tissue development in vitro, ex vivo, and/or in vivo without inducing any adverse effects. The isolated ECM is substantially free of immunogenic components such that it can be administered to a subject. The isolated ECM is substantially free of cellular and nuclear debris and is substantially clean and has reduced immunogenicity, such that it can be used as a biomaterial. The ECM is substantially free of cellular debris and nuclease debris, such as DNA.

The structural and functional properties of a native ECM are intact in the isolated ECM. The components of the ECM for wound healing and treating and preventing skin conditions are retained in the isolated ECM.

The present disclosure describes ECM compositions and composites including the isolated ECM. In addition to the isolated ECM, the composition can include one or more carriers or excipients. Examples of carriers and excipients include saline, emulsion, a mixture of organic solvents with water, calcium carbonate, calcium phosphate, various sugars and types of starch, cellulose derivatives, gelatin, vegetable oils, petrolatum, lanolin, mineral oil, dimethicone, humectant, and polyethylene glycols. Examples of humectants include glycerin, lecithin, and propylene glycol. In embodiments, the compositions described herein include cosmetic, or pharmaceutical compositions containing one or more cosmetically, or pharmaceutically acceptable carriers or excipients, respectively.

The compositions described herein can include one or more agents such as one or more therapeutic agents. Examples of therapeutic agents include known drugs such as retinoic acid, corticosteroids, antifungals, antivirals, antibiotics, antiseptics, local anesthetics, and antineoplastics.

The compositions described herein can include one or more agents such as one or more cosmetic agents. Examples of cosmetic agents include antioxidants, peptides, alpha or beta hydroxy acids, retinol, vitamins, plant extracts, skin clarifying agents such as arbutin, moisturizing agents such as hyaluronic acid, emollients, carbohydrates, glycoproteins, and/or polymers. The one or more agents can include a combination of agents. The agent can be exogenous or xenogenic to the isolated ECM.

Examples of one or more peptides and proteins include growth factors, cytokines, and chemokines. Examples of growth factors include fibroblast growth factors (FGFs) including acidic FGF, basic FGF, FGF8, and FGF10; ciliary neurotrophic factor (CNTF); epidermal growth factor (EGF); granulocyte-macrophage colony stimulating factor (GM-CSF); hepatocyte growth factor (HGF); insulin-like growth factors 1 and 2 (IGF-1 and IGF-2); keratinocyte growth factor (KGF); nerve growth factor (NGF); neurotrophins such as neurotrophin-3, neurotrophin-4, neurotrophin-5; platelet-derived growth factor (PDGF); vascular endothelial growth factor (VEGF); stromal-derived factor 1 alpha (SDF-1 alpha); and transforming growth factor-alpha and -beta (TGF-α and TGF-β). Examples of cytokines and chemokines include tumor necrosis factor-alpha (TNF-α), interleukin-1 alpha and beta (IL-1α and IL-1β), interleukin-6 (IL-6), interleukin-7 (IL-7), interleukin-18 (IL-18), CCL2, CCL3, CCL5, CXCL1, CXCL4, CXCL5, CXCL7, CXCL8, and CXCL12. Examples of cosmetic peptides include acetyl hexapeptide, acetyl tetrapeptide, palmitoyl pentapeptide, and palmitoyl oligopeptide.

Examples of one or more therapeutic agents include antimicrobials and anti-inflammatory agents. Examples of antimicrobials include antibiotics such as penicillin, streptomycin, amoxicillin, cephalexin, clindamycin, dicloxacillin, and doxycycline. Other antimicrobials include anti-microbial peptides, silver salts, clotrimazole, miconazole, and ketoconazole. Examples of anti-inflammatory agents include nonsteroidal anti-inflammatory drugs (NSAIDs) such as salicylic acid, ibuprofen, naproxen, colchicine, fenoprofen, sulindac, diflunisal, diclofenac, indoprofen, and sodium salicylamide.

Examples of cosmetic agents include one or more glycoproteins include proteoglycans which are proteins covalently attached to glycosaminoglycans (GAGs), antioxidants, ascorbic acid, vitamin C, alpha hydroxy acids (AHAs), beta hydroxy acids (BHAs), exfoliants, skin whitening agents, light diffusers, UV absorbing agents, sunscreens, moisturizers, anti-wrinkle ingredients, and oil absorbing agents. Examples of AHAs include glycolic acid, lactic acid, malic acid, tartaric acid, and citric acid. Examples of BHAs include salicylic acid.

The compositions described herein can also include one or more natural and/or synthetic polymers. Natural polymers can be from an animal source or a non-animal source such as a plant source. Examples of natural polymers include natural polymers such as collagen, chitosan, alginate, glycosaminoglycans, fibrin, and hyaluronic acid. Examples of synthetic polymers include polyethylene, polyethylene glycol (PEG), polyethylene terephthalate (PET, or PETE), polytetrafluoroethylene (PTFE), polylactic acid (PLA), polyglycolic acid (PGA), polyethylene glycol (PEG), poly(ethylene glycol) diacrylate (PEG diacrylate), poly(hydroxy acids), polydioxanone, polycaprolactone, poly(ortho esters), poly(anhydrides), polyphosphazene, poly(amino acids), pseudo-poly(amino acids), conductive polymers (such as polyacetylene, polypyrrole, polyaniline), polyurethane, polystyrene, and nitinol.

The polymer of the compositions described herein can be biocompatible, biodegradable, and/or bioabsorbable, and can be a random copolymer, block copolymer, or blend of monomers, homopolymers, copolymers, and/or heteropolymers that contain these monomers. Exemplary biodegradable or bioabsorbable polymers include polylactides, poly-glycolides, polycaprolactones, polydioxanes, and their random and block copolymers. A biodegradable and/or bioabsorbable polymer can contain a monomer selected from the group consisting of glycolide, lactide, dioxanone, caprolactone, trimethylene carbonate, ethylene glycol, and lysine. The biodegradable and/or bioabsorbable polymers can contain bioabsorbable and biodegradable linear aliphatic polyesters such as polyglycolide (PGA) and its random copolymer poly (glycolide-co-lactide-) (PGA-co-PLA). Other examples of suitable biocompatible polymers include polyhydroxyalkyl, methacrylate, ethylmethacrylate, polyvinylpyrrolidone, and polyacrylamides. Other suitable bioabsorbable materials are biopolymers which include collagen, gelatin, alginic acid, chitin, chitosan, fibrin, hyaluronic acid, dextran, polyamino acid, polylysine, and copolymers of these materials. Any combination of polymers and copolymers or blend thereof of the above examples can also be included in the composition.

The compositions described herein can also include protectives, adsorbents, demulcents, emollients, preservatives, antioxidants, moisturizers, buffering agents, solubilizing agents, skin-penetration enhancers, and surfactants.

Any skin penetration enhancer can be added to the compositions, provided the skin penetration enhancer is safe and can effectively facilitate the passage of the desired substances in the isolated ECM across the skin membrane. Examples of skin penetration enhancers include dimethyl sulphoxide (DMSO), monoglycerides, C10-C20 fatty acid esters including ethyl palmitate and isopropyl myristate; acyl lactylates such as caproyl lactylic acid and lauroyl lactylic acid; dimethyl lauramide; dodecyl (lauryl) acetate; lactate esters such as lauryl lactate, and myristyl lactate; monoalkyl ethers of polyethyleneglycol and their alkyl or aryl carboxylic acid esters and carboxymethyl ethers such as polyethylene glycol-4 lauryl ether (Laureth-4) and polyethylene glycol-2 lauryl ether (Laureth-2); Myreth-3, myristyl sarcosine, and methyl laurate; polypropylene glycol, polyethylene glycol, lecithin, urea, amino acids, 1-dodecylhexahydro-2H-azepine 2-one (Azone), oleic acid, linoleic acid, isopropyl linoleate, oleyl alcohol, 1-dodecyl-azacycloheptan-2-one, butanediol, and 2-(2-Ethoxyethoxy)ethanol (Transcutol).

The isolated ECM and the compositions described herein can be prepared as a dry powder, a solution, a paste, a liquid, an extract, a cream, a lotion, a serum, an emollient, an ointment, a dispersion, or an emulsion. The isolated ECM and the compositions can be prepared into a variety of suitable shapes and sizes as they can be formed, laminated, homogenized, and reconstituted. They can be formed into two-dimensional or three-dimensional shapes. They can be formed into a sheet, a mesh, a graft, a plug, or any shape or form for use. The sheets can include backing with or without an adhesive. The backing can be biodegradable or non-biodegradable. Two or more sheets can be laminated together or somehow attached. The sheets can be oriented in the same direction, different direction, or at an angle. There can be two to fifteen layers of sheets. The sheets can be from different sources of amphibian ECM.

In embodiments, the powder containing the micronized particulates can be reconstituted with water, a buffered solution, or any suitable liquid and/or carriers to form a solution, a paste, a liquid, an extract, a cream, a lotion, a serum, an emollient, an ointment, a dispersion, or an emulsion. The solution can be a buffered solution, similar to that used for the decellularization process.

The isolated ECM described herein has been shown to have therapeutic or cosmetic activity dependent on the degree of decellularization, concentration, and/or final composition. Accordingly, the isolated ECM therapeutic compositions described herein can be used in vitro, ex vivo, and/or in vivo. Because the isolated ECM has various bioactivities which are involved in regenerative cellular processes, the isolated ECM and the compositions described herein can be used as a biomaterial for wound healing, regeneration of tissue, scaffolding for growing cells, and treating and preventing various skin conditions. The biomaterial can be formed as an implant for regenerating tissues. Since the isolated ECM can also serve as a scaffold, cells can grow on the ECM to enhance the regeneration and wound healing of tissues and organs. The biomaterial can also be applied to, coated on, and/or infused in an implant or a medical device for administering to a subject. The medical device can be a patch, a bandage, or any suitable device for delivering the biomaterial. Thus, the biomaterial can be used as a material for any xenogenic transplantation or xenograft.

The biomaterial can also serve as a system or device for delivering agents to tissues and organs for wound healing, tissue regeneration, and treating and/or preventing various skin conditions. The endogenous agents, such as growth factors in the biomaterial, can be delivered via immediate release or controlled release. Moreover, exogenous or xenogenic agents described added to the biomaterial can be delivered via immediate release or controlled release.

Carriers for immediate or sustained release preparations include polymers. The polymers can be biodegradable, and/or bioabsorbable. As an example, for controlled release, the biomaterial can be coated with polymers such as acrylic polymer, acrylic/methacrylic copolymer, cellulose acetate phthalate (CAP), Opadry®, and Ethocel™. For immediate release, the biomaterial can be coated with cellulosic polymers, such as hydroxypropyl methylcellulose (HPMC), hydroxypropyl cellulose (HPC), hydroxyethylcellulose (HEC), methyl cellulose (MC), and sodium carboxymethyl cellulose (NaCMC); vinyl derivatives, such as polyvinyl pyrrolidone (PVP), polyvinyl pyrrolidone-polyvinyl acetate copolymer, polyvinyl alcohol (PVA), and polyvinyl alcohol-polyethylene glycol copolymer; acrylic polymers, such as Eudragit®; or glycols such as polyethylene glycols.

The isolated ECM and the compositions described herein can be prepared as a formulation for treating and/or preventing various skin conditions including inflammatory and cancerous skin conditions. The formulation can be in the form of a dry powder, a solution, a paste, a liquid, an extract, a cream, a lotion, a serum, an emollient, an ointment, a dispersion, or an emulsion. The amount of isolated ECM in the formulation is 0.001% to 5.0% w/v, 0.01% 4%, or 0.05% to 1.0% w/v. The formulation can be placed on or impregnated into a bandage, such as a non-stick adhesive bandage, for applying to the subject.

Examples of skin conditions requiring therapeutic treatment include acne, actinic keratosis, blister, cellulitis, cold blister, hives, impetigo, Keratosis pilaris, melasma, moles, ringworm, uticaria, vitiligo, and wart. Examples of inflammatory skin conditions include psoriasis; dermatitis, such as contact dermatitis, atopic dermatitis (eczema), seborrheic dermatitis, nummular dermatitis, generalized exfoliative dermatitis, statis dermatitis, lichen simplex chronicus; disorders of hair follicles and sebaceous glands, such as acne, rosacea and rhinophyma, perioral dermatitis, and pseudofolliculitis barbae; and inflammatory reactions, such as drug eruptions, erythema multiforme, erythema nodosum, and granuloma annulare. Examples of cancerous skin conditions include basal cell carcinoma, melanoma, and squamous cell carcinoma. Other skin conditions needing treatment include fine lines and/or wrinkles, aging, redness, abrasion, burn, cut, infection, razor bumps, scars, uneven skin tone, pain, stretch marks, skin elasticity and/or firmness, skin hydration, and hyperpigmentation. The burns include acute thermal burns including first, second, or third-degree burns.

The isolated ECM and the compositions described herein can also be used in a skincare regimen for protecting the skin from damage including UV rays and environmental pollution and as an aesthetic agent for improving the appearance of the skin.

The isolated ECM and compositions described herein can be administered to the target site topically, or by injection, implantation, microneedling, or radiofrequency microneedling, or using an ablative fractional laser. The isolated ECM and compositions described herein can be administered to the subject prior to, during, or after a dermatological or cosmetic procedure, such as dermabrasion, microdermabrasion, and ablative laser resurfacing. The dermatological or cosmetic procedure includes procedures wherein at least one cell of the stratum corneum is removed. The isolated ECM and compositions described herein can also be delivered as an injectable or with a dermal or subdermal implant such as a volume filler, hyaluronic acid, or other dermal matrix protein including collagen or elastin, either naturally occurring, bioengineered, or recombinantly produced. The isolated ECM and compositions described herein can be administered alone or in combination with one or more agents described herein, such as growth factors, peptides, or proteins. The isolated ECM can also be administered with a toxin, such as botulinum toxin.

The isolated ECM and compositions described herein can reduce inflammation, reduce scarring, reduce keloid formation, reduce or diminish the severity of scarring and keloid formation, and/or reduce healing time for various dermatological and cosmetic procedures. The isolated ECM and compositions described herein can also be used to restore lost dermal matrix or subdermal volume.

The present disclosure describes kits including the isolated ECM or compositions described herein for the various uses described herein. The kits can include sterilized isolated ECM or a composition thereof in any shape and form. The kits can include a solution for reconstituting the ECM for use. The kits can include a device for administering the isolated ECM or composition to a subject. The kits can include an implant to be coated with the ECM or composition prior to being implanted in a subject. The kits can include components for the various uses described herein.

The present disclosure also describes a medical device comprising the isolated ECM or compositions described herein for the various uses described herein. A medical device can be a material or an object used directly or indirectly to apply the isolated ECM or compositions described herein. As an example, a medical device can be used to apply the isolated ECM or compositions described herein on the skin of a subject.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Numbers expressing ranges or quantities of ingredients, constituents, reaction conditions, and so forth used in the specification and claims are to be understood as being modified by the term “about.” When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±15% of the stated value; ±10% of the stated value; ±5% of the stated value; ±4% of the stated value; ±3% of the stated value; ±2% of the stated value; ±1% of the stated value; or ±any percentage between 1% and 20% of the stated value.

As will be understood by one of ordinary skill in the art, each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. In embodiments, the lack of a material effect of a step is evidenced by the lack of a statistically-significant reduction in the process step in removing cellular debris such as DNA from the sample. Lack of a material effect of an embodiment can include the lack of a statistically-significant improvement in using the isolated ECM in wound healing, cell growth, cell repopulation, cell attachment, cell proliferation, or cell migration.

Recitation of ranges of values herein is merely intended to serve as a shorthand method of referring individually to each separate value falling within the range. Unless otherwise indicated herein, each individual value is incorporated into the specification as if it were individually recited herein. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.5, 2.7, 3, 4, 5, 5.1, 5.3, 5.8 and 6. Moreover, any ranges cited herein are inclusive.

Groupings of alternative elements or embodiments of the invention disclosed herein are not to be construed as limitations. Each group member may be referred to and claimed individually or in any combination with other members of the group or other elements found herein.

The following exemplary embodiments and examples illustrate exemplary methods provided herein. These exemplary embodiments and examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### Exemplary Embodiments

The following are exemplary embodiments:
1. An isolated amphibian ECM, wherein the ECM is decellularized, includes an intact native ECM structure, and is characterized by having:
   less than 1% to 10% of the total DNA present in a corresponding native ECM sample;
   residual DNA fragments of less than 300 bases (bps); and/or
   greater than 70% of its cells removed.
2. The ECM of embodiment 1, wherein the ECM is characterized as having less than 1%, 2% to 9%, 2% to 5%, 3% to 8%, 3% to 7%, 3% to 6%, 3% to 5%, 3% to 4%, 2% to 3%, 4% to 5%, 5% to 6%, 6% to 7%, 7% to 8%, 1%, 2%, 2.5%, 3%, 3.5%, 4%, 4.5%, 5%, 5.5%, 6%, 6.5%, 7%, 7.5%, or 8.0% of the total amount DNA present in the corresponding native ECM sample.
3. The ECM of embodiment 1 or 2, wherein the ECM is characterized as having residual DNA fragments of less than 50 bps, 50 to 300 bps, 60 to 290 bps, 70 to 280 bps, 80 to 270 bps, 90 to 260 bps, 100 to 250 bps, 100 to 240 bps, 100 to 230 bps, 100 to 220 bps, 100 to 210 bps, or 100 to 200 bps in length.
4. The ECM of any one of embodiments 1-3, wherein the ECM is characterized by having residual DNA fragments of less than 250 bps in length.
5. The ECM of any one of embodiments 1-4, wherein the ECM is characterized by having greater than 99%, 95% to 99%, 90% to 99%, 71% to 99%, 75% to 95%, 80% to 90%, or 80% to 85% of its cells removed.
6. The ECM of any one of embodiments 1-5, wherein the ECM is in the form of dry powder, a paste, a solution, an extract, a cream, a lotion, dispersion, or an emulsion.
7. The ECM of any one of embodiments 1-6, wherein the ECM is obtained from a young amphibian, optionally the young amphibian includes froglet, tadpole, Urodele, or larval stage young Apoda.
8. The ECM of any one of embodiments 1-7, wherein the ECM is obtained from a neotenic Urodele.
9. A composition including the ECM of any one of embodiments 1-8 and a carrier.
10. The composition of embodiment 9, wherein the composition is a pharmaceutical or cosmetic composition, and optionally wherein the pharmaceutical composition includes a pharmaceutically acceptable carrier, and wherein the cosmetic composition includes a cosmetically acceptable carrier.
11. The composition of embodiment 9 or 10, wherein the composition further includes one or more agents, and optionally, wherein the one or more agents are xenogenic to the isolated ECM.
12. The composition of any one of embodiments 9-11, wherein the composition further includes one or more agents that are peptides, proteins, drugs, nutrients, retinoids, emollients, steroids, carbohydrates, glycoproteins, polymers, or a combination thereof.
13. The composition of any one of embodiments 9-12, wherein the composition further includes one or more growth factors, cytokines, chemokines, or a combination thereof.
14. The composition of any one of embodiments 9-13, wherein the composition further includes one or more polymers, and optionally wherein the one or more polymers are synthetic or natural polymers or copolymers.
15. A method of decellularizing amphibian ECM, wherein the method includes harvesting a biological sample from an amphibian, washing the sample with a detergent solution, and treating the sample with one or more proteases and one or more nucleases to obtain an isolated ECM.
16. The method of embodiment 15, wherein the method includes harvesting a biological sample from an amphibian, rinsing the sample with a solution, washing the sample with a detergent solution, treating the sample with a protease, rinsing the sample after the protease treatment, and treating the rinsed protease treated sample with nuclease, to obtain an isolated ECM, and optionally, further washing the isolated ECM.
17. The method of embodiment 15 or 16, wherein the method includes rinsing with water or buffered solution, optionally phosphate buffered saline (PBS) or Dulbecco's phosphate buffered saline (DPBS).
18. The method of any one of embodiments 15-17, wherein the sample is washed with a detergent solution including one or more surfactants.
19. The method of any one of embodiments 15-18, wherein the sample is washed with a detergent solution including one or more non-ionic surfactant or an anionic surfactant.
20. The method of any one of embodiments 15-19, wherein the sample is washed with a detergent solution including 2-[4-(2,4,4-trimethylpentan-2-yl)phenoxy]ethanol, polyoxyethylene (10) isooctylcyclohexyl ether, polyglycol ether, or trimethylnonylpolyethylene glycol.
21. The method of any one of embodiments 15-19, wherein the sample is washed with a detergent solution including N-lauryl sarcosine (NLS), potassium lauryl sarcosine, cholate, deoxycholate, sodium dodecyl sulfate (SDS), or polyoxyethylene sorbitan fatty acid ester, optionally Tween, 20, 40, 60, or 80.
22. The method of any one of embodiments 15-21, wherein the sample is incubated with one or more proteases including one or more amino-endopeptidases.
23. The method of any one of embodiments 15-22, wherein the sample is incubated with one or more proteases including dispase I, dispase II, trypsin, papain, and collagenase.
24. The method of any one of embodiments 15-23, wherein the sample is incubated with one or more nucleases including a non-specific DNA/RNA endonuclease.
25. The method of any one of embodiments 15-24, wherein the sample is incubated with one or more nucleases including benzonuclease, turbonuclease, pulmozyme, and a combination of DNase and RNase, optionally recombinant DNases and RNase.
26. The method of any one of embodiments 15-25, wherein the method includes the following steps in the following order:
   harvesting a biological sample from an amphibian;
   rinsing the sample with buffered saline;
   washing the sample with a detergent solution including 2-[4-(2,4,4-trimethylpentan-2-yl)phenoxy]ethanol or NLS;
   rinsing the sample with buffered saline;
   treating the rinsed sample with dispase Il;
   rinsing the sample with buffered saline; and
   treating the rinsed sample with benzonuclease to obtain an isolated ECM;
and optionally, the isolated ECM is washed with DPBS.
27. The method of any one of embodiments 15-26, wherein the sample is washed with the detergent solution for 6 hours with shaking.
28. The method of any one of embodiments 15-27, wherein in between washing with the detergent solution and treating with protease and between treating with protease and treating with nuclease, the sample is rinsed with DPBS for 5 minutes.
29. The method of any one of embodiments 15-28, wherein the amphibian includes froglet, tadpole, Urodele, or larval stage young Apoda.
30. The method of any one of embodiments 15-29, wherein the biological sample includes ECM tissue from a neotenic Urodele.
31. The method of any one of embodiments 15-30, wherein the biological sample includes ECM of connective tissues, adipose tissue, bone, blood plasma, skin, cartilage, tendon, dura mater, and fascia.
32. The method of any one of embodiments 15-32, wherein the isolated ECM is further micronized.
33. The method of embodiment 32, wherein the isolated ECM can be micronized to a particle size of about 1 to 1000 microns, 1 to 900 microns, 1 to 800 microns, 1 to 700 microns, 1 to 600 microns, 1 to 500 microns, 1 to 400 microns, 1 to 300 microns, 1 to 200 microns, 100 to 300 microns, 100 to 250 microns, 100 to 200 microns, 100 to 150 microns, or 100 to 125 microns.
34. An isolated ECM obtained by the method of any one of embodiments 15-33.
35. A composition comprising the isolated ECM of embodiment 34 and a carrier.
36. The composition of embodiment 35, wherein the composition is a pharmaceutical or cosmetic composition, and optionally wherein the pharmaceutical composition includes a pharmaceutically acceptable carrier, and wherein the cosmetic composition includes a cosmetically acceptable carrier.
37. The composition of embodiment 35 or 36, wherein the composition further includes one or more agents, and optionally, wherein the one or more agents are xenogenic to the isolated ECM.
38. The composition of any one of embodiments 35-37, wherein the composition further includes one or more agents that are peptides, proteins, drugs, nutrients, retinoids, emollients, steroids, carbohydrates, glycoproteins, polymers, or a combination thereof.
39. The composition of any one of embodiments 35-38, wherein the composition further includes one or more growth factors, cytokines, chemokines, or a combination thereof.
40. The composition of any one of embodiments 35-39, wherein the composition further includes one or more polymers, and optionally wherein the one or more polymers are synthetic or natural polymers or copolymers.
41. A method of treating and/or preventing a skin condition, wherein the method includes administering the isolated ECM of any one of embodiments 1-8 or 34 or the composition of any one of embodiments 9-14 or 35-40 to a subject in need thereof.
42. The method of embodiment 41, wherein the skin condition includes fine lines and/or wrinkles, aging, redness, abrasion, burns, cuts, infection, razor bumps, scars, uneven skin tone, pain, stretch marks, increasing skin elasticity and/or firmness, improve skin hydration, inflammation, and hyperpigmentation.
43. The method of embodiment 41 or 42, wherein preventing a skin condition includes protecting the skin from damages including UV rays and/or environmental pollution.
44. The method of any one of embodiments 41-43, wherein treating and/or preventing skin condition includes a skin condition after a dermatological or cosmetic procedure, optionally, the procedure comprises dermabrasion, microdermabrasion, or ablative surface resurfacing.
45. The method of any one of embodiments 41-44, wherein treating and/or preventing a skin condition includes using the isolated ECM to improve the appearance of the skin of a subject.
46. A formulation including the isolated ECM of any one of embodiments 1-8 or 34 or the composition of embodiments 9-14 or 35-40.
47. The formulation of embodiment 46, wherein the formulation is in the form of a powder, a solution, a paste, a liquid, an extract, a cream, a lotion, a serum, a dispersion, an emulsion, or an ointment.
48. The formulation of embodiment 46 or 47, wherein the formulation includes 0.001% to 5.0% w/v of the isolated ECM.

### EXAMPLES

### Example 1. Harvesting a Skin Sample from Axolotl

A Urodele skin sample is obtained or harvested from a neotenic Urodele. A full-thickness skin explant sample of 5 by 5 centimeters (cm) of skin is surgically removed from a neotenic Urodele. The sample is cleaned and prepared for decellularization.

### Example 2. Decellularization of the Harvested Axolotl Skin Sample

Fresh axolotl samples were imaged to document appearance prior to processing. All samples were weighed before and after every step to record weight loss due to decellularization (Table 1).

Washing. Three 5 by 5 cm samples were rinsed briefly for 5 minutes (mins) with Dulbecco's Phosphate buffered saline (DPBS) at room temperature, followed by washing with Triton X-100 Reduced or NLS for 6 hrs, at 37° C., and subsequently briefly rinsed with DPBS for 5 mins at room temperature before being incubated with one or more proteases. The washes with the detergent solution were performed with shaking at 100 RPM.

Triton X-100 Reduced Wash (Samples T1 and T2): Following the rinse with PBS, each of the 5 by 5 cm samples was washed twice with 20 ml of 0.5% v/v Triton X-100 Reduced containing 0.02% w/v sodium azide, for 6 hrs each time at 37° C. with shaking at 100 RPM.

NLS Wash (Sample NLS): Following the rinse with PBS, each of the 5 by 5 cm samples was washed twice with 25 ml of 1.0% NLS containing 0.02% sodium azide for 6 hrs each time at 37° C.

Protease Treatment. The samples were each incubated with 2 units/ml of dispase II (2 units of dispase II per ml of high glucose DPBS) for 90 mins at 37° C. The protease treated samples were each briefly rinsed with DPBS for 5 minutes at room temperature.

Nuclease Treatment: After rinsing with DPBS, the protease treated samples were then treated with 40 units/ml of benzonuclease (40 units of benzonuclease per ml of DPBS) at 37° C. for 6 hrs. Subsequently, the samples were briefly rinsed with DPBS for 5 minutes at room temperature and washed with DPBS for 24 hrs at 37° C.

### Example 3. Analysis of Isolated ECM

Hematoxylin and Eosin (H&E) Staining. After decellularization was completed, two (2) 8 mm punch biopsy samples, one from the left top corner and one from the center of each 5 by 5 sample, were taken from each of the three samples to compare the success of the decellularization process based on anatomical area.

H&E slides were processed and imaged via light microscopy (FIGS. 1A-1F). Decellularization degree above 80% was achieved as assessed by visual inspection.

DNA Quantitation. To quantify the DNA in the isolated ECM after the decellularization process, 4 mm punch biopsy samples were excised from two different locations, the bottom left corner and center of each 5 by 5 sample, from each of the three samples. Immediately after excision, the samples were transferred to Eppendorf tubes and frozen at −20° C. until ready for DNA extraction and quantification. DNA was extracted and purified with a DNEasy Blood & Tissue Kit. Samples were subjected to the protocol included in the kit and subsequently quantified with a NanoDrop One Instrument.

The results shown in the graphs of FIGS. 2A and 2B indicate the proper removal of DNA by benzonase treatment. In FIG. 2A, samples collected from the tissue center (C) for T1 (T1_C), T2 (T2_C), and NLS (NLS_C) yielded DNA concentrations of 4.44 ng/μl±0.9, 3.96 ng/μl±0.83, and 3.27 ng/μl±0.52, respectively (ng of DNA per ul of DNA extract). Samples collected from the bottom left corner (BLC) of T1, NLS, and T2 yielded DNA concentrations of 7.44 ng/μl±3.88, 5.07 ng/μl±0.61, 4.3 ng/μl±0.48 for T1_BLC, T2_BLC, and NLS_BLC, respectively. FIG. 2B shows the concentration of DNA converted into a percentage.

DNA Gel Electrophoresis. DNA quantification was completed via gel electrophoresis and shown in FIGS. 3A and 3B. Samples were prepared with Purple 6× gel loading dye (New England BioLabs, B7024S) at a 5:1 ratio (DNA:Stain). A DNA ladder (GeneRuler Express DNA Ladder Ref #SM1553) was used for reference. A total volume of 12 μL (10 μL of DNA sample+2 μL of Purple Dye) was used per well for samples as well as for the DNA ladder. Agarose gel (1%) with GelRed Nucleic Acid Stain (MilliporeSigma SCT123) was used to run the samples at 100 volts for 45 minutes. Subsequently, the gel was removed and imaged with an Azure Biosystems c150 gel imaging device. As shown in FIGS. 3A and 3B, faint DNA bands were observable in the samples tested suggesting that the benzonase treatment was effective.

Example 4. Micronization of Isolated ECM

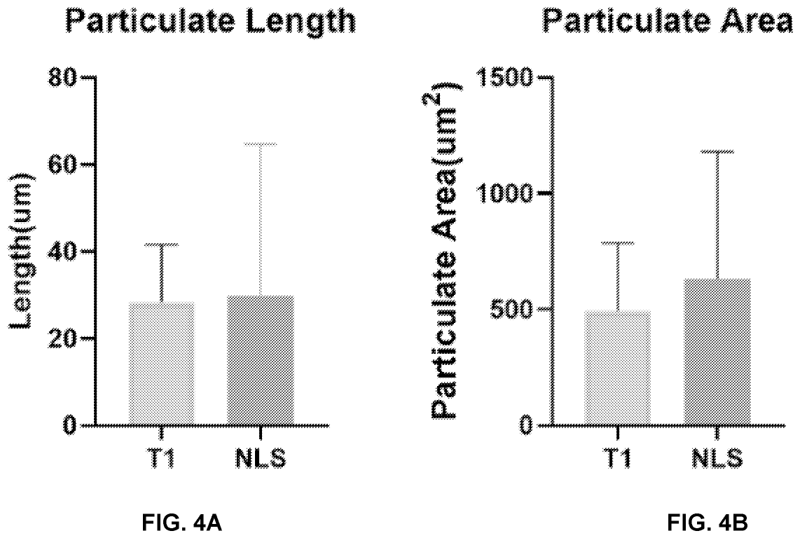

Cryomilling. The T1 and NLS samples were micronized by cryomilling to obtain particulates. The samples were cryomilled under the following conditions in a Spex 6870 Freezer/Mill. First, samples were flash-frozen in liquid nitrogen without direct exposure. Subsequently, 3 ml of RNA/DNA-free water (Thermo-Scientific, Water, nuclease-free Ref#R0582) was added 1 ml at a time into the cryomilling chamber containing the sample. Immediately after, the bullet was introduced into the cryomilling apparatus and ran at 5 minutes pre-cool, 1 minute cryomilling, 2 minutes cooling, and 6 collisions per second for a total of 6 cycles. A small sample of the slurry was retained for visual examination under light microscopy. Additionally, 15 randomly selected micronized tissue samples were measured via ImageJ and tabulated to assess particulate length (fiber-like structures) and area (patches). The Triton X-100 treated sample yielded fiber lengths of 28.36 μm±13.19 in comparison to 30.05 μm±34.70 for NLS treated sample (FIG. 4A). The average area of the micronized T1 and NLS samples were 494.06 μm²±293.2 and 631.5 μm²±550.91, respectively. Moreover, fiber size distribution was more homogenous on Triton X-100 treated samples than the NLS treated sample. The results provide evidence that the cryomilling process and settings are effective at micronizing axolotl decellularized tissue to sub-100 um in length.

The T2 sample was not micronized but was dehydrated under vacuum at 15° C. and 360 Torr.

Lyophilization. Immediately after cryomilling, the T1 and NLS samples were transferred into 50 ml conical tubes for lyophilization. The process for lyophilization is as follows. The sample is frozen at −45° C. for 5 minutes at 360 Torr. Subsequently, the sample underwent a primary drying phase which includes freezing at −40° C. for 120 minutes at 100 mTorr, followed by freezing at −25° C. for 300 minutes at 50 mTorr, and freezing at −25° C. for 720 minutes at 250 mTorr. Finally, the samples underwent a second drying phase which includes cooling at +5° C. 120 minutes at 300 mTorr.

The subject matter described above is provided by way of illustration only and should not be construed as limiting. Various modifications and changes may be made to the subject matter described herein without following the example embodiments and applications illustrated and described, and without departing from the true spirit and scope of the present disclosure, which is set forth in the following claims.

### Example 5. Treatment of Subjects

### Example 5.1. Topical Serum for Treating a Cut

A topical serum formulation is prepared to contain between 0.001% to 5.0% weight by volume of the isolated ECM and one or more carriers. The topical serum is applied twice daily in the morning and at night to the site of the cut on the subject. At 8 weeks, the appearance of skin quality is observed.

### Example 5.2. Topical Treatment of a Skin Abrasion

A formulation is prepared to contain between 0.001% to 5.0% weight by volume of the isolated ECM in a petrolatum base and optionally one or more other carriers. The formulation is placed in a non-stick adhesive bandage. The bandage was applied to the subject at the site of the abrasion. The bandage is changed daily and the site of abrasion is observed to determine the number of days it takes for the skin to re-epithelialize.

For comparison, an abrasion of a subject is treated with only petrolatum. The site of abrasion is observed to determine the number of days it takes for the skin to re-epithelialize.

### Example 5.3. Topical Treatment After Ablative Laser Resurfacing Procedure

A formulation is prepared to contain between 0.001% to 5.0% weight by volume of the isolated ECM and one or more carriers or excipients. The formulation is applied daily to the skin of the subject after undergoing an ablative laser resurfacing procedure. The skin is observed daily for reduced inflammation and shorter healing time.

### Example 5.4. Topical Treatment for Treating a Burn

A formulation is prepared to contain between 0.001% to 5.0% weight by volume of the isolated ECM in a petrolatum base and optionally one or more other carriers. A subject presented with a burn to the body is treated with the formulation. The formulation is placed on a dressing and applied at the site of the burn on the subject. The burn wound is checked daily, and the dressing is changed daily or every 48 hrs.

All publications, patents, and patent applications cited in this specification are incorporated herein by reference in their entirety as if each individual publication, patent, or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

U.S. Pat. No. 10,617,790

1. An isolated amphibian ECM, wherein the ECM is decellularized and comprises
an intact native ECM structure;
only 1% to 10% or 2% to 10% of the total DNA present in a corresponding native ECM sample;
residual DNA fragments of less than 300 bases (bps) in length, 50 bps to 300 bps in length, or less than 250 bps in length; and/or
greater than 70% or 80% to 90% of its cells removed.

2.-5 (canceled)

6. The ECM of claim 1, wherein the ECM is in the form of dry powder, a paste, a solution, an extract, a cream, a lotion, dispersion, or an emulsion.

7. The ECM of claim 1, wherein the ECM is obtained from a young amphibian, optionally the young amphibian comprises froglet, tadpole, Urodele, or larval stage young Apoda.

8. The ECM of claim 1, wherein the ECM is obtained from a neotenic Urodele.

9. A composition, wherein the composition comprises the ECM of claim 1 and a carrier.

10. The composition of claim 9, wherein the composition is a pharmaceutical or cosmetic composition, and optionally wherein the pharmaceutical composition comprises a pharmaceutically acceptable carrier, and wherein the cosmetic composition comprises a cosmetically acceptable carrier.

11. The composition of claim 10, wherein the composition further comprises one or more agents, and optionally, wherein the one or more agents are xenogenic to the isolated ECM.

12. The composition of claim 10, wherein the composition further comprises one or more agents comprising peptides, proteins, drugs, nutrients, retinoids, emollients, steroids, carbohydrates, glycoproteins, polymers, or a combination thereof.

13. The composition of claim 10, wherein the composition further comprises one or more growth factors, cytokines, chemokines, or a combination thereof.

14. The composition of claim 9, wherein the composition further comprises one or more polymers, and optionally wherein the one or more polymers are synthetic or natural polymers or copolymers.

15. A method of decellularizing amphibian ECM, wherein the method comprises harvesting a biological sample from an amphibian, washing the sample with a detergent solution, and treating the sample with one or more proteases and one or more nucleases to obtain an isolated ECM.

16. The method of claim 15, wherein the method comprises harvesting a biological sample from an amphibian, rinsing the sample with a solution, washing the sample with a detergent solution, treating the sample with a protease, rinsing the sample after the protease treatment, and treating the rinsed protease treated sample with nuclease, to obtain an isolated ECM, and optionally, further washing the isolated ECM.

17. The method of claim 16, wherein the method comprises rinsing the sample with water or buffered solution, optionally phosphate buffered saline (PBS) or Dulbecco's phosphate buffered saline (DPBS).

18. The method of claim 16, wherein the sample is washed with a detergent solution comprising one or more surfactants, and optionally wherein the one or more surfactants comprises one or more non-ionic surfactant or an anionic surfactant.

19. The method of claim 16, wherein the sample is washed with a detergent solution comprising 2-[4-(2,4,4-trimethylpentan-2-yl)phenoxy]ethanol, polyoxyethylene (10) isooctylcyclohexyl ether, polyglycol ether, or trimethylnonylpolyethylene glycol.

20. The method of claim 16, wherein the sample is washed with a detergent solution comprising N-lauryl sarcosine (NLS), potassium lauryl sarcosine, cholate, deoxycholate, sodium dodecyl sulfate (SDS), or polyoxyethylene sorbitan fatty acid ester, and optionally Tween, 20, 40, 60, or 80.

21. The method of claim 16, wherein the sample is incubated with one or more proteases comprising one or more amino-endopeptidases, and optionally wherein the one or more proteases comprises one or more proteases comprising dispase I, dispase II, trypsin, papain, and collagenase.

22. The method of claim 16, wherein the sample is incubated with one or more nucleases comprising a non-specific DNA/RNA endonuclease, and optionally wherein the one or more nucleases comprises benzonuclease, turbonuclease, pulmozyme, and a combination of DNase and RNase, optionally recombinant DNases and RNase.

23. The method of claim 16, wherein the method comprises the following steps in the following order:
harvesting a biological sample from an amphibian;
rinsing the sample with buffered saline;
washing the sample with a detergent solution comprising 2-[4-(2,4,4-trimethylpentan-2-yl)phenoxy]ethanol or NLS;
rinsing the sample with buffered saline;
treating the rinsed sample with dispase Il;
rinsing the sample with buffered saline; and
treating the rinsed sample with benzonuclease to obtain an isolated ECM;
and optionally, the isolated ECM is washed with DPBS.

24. The method claim 23, wherein the sample is washed with the detergent solution for 6 hours with shaking.

25. The method of claim 23, wherein in between washing with detergent solution and treating with protease and in between treating with protease and treating with nuclease, the sample is rinsed with DPBS for 5 minutes.

26. The method of claim 16, wherein the amphibian comprises froglet, tadpole, Urodele, or larval stage young Apoda.

27. The method of claim 16, wherein the biological sample comprises ECM tissue from a neotenic Urodele.

28. The method of claim 16, wherein the biological sample comprises ECM of connective tissues, adipose tissue, bone, blood plasma, skin, cartilage, tendon, dura mater, or fascia.

29. The method of claim 16, wherein the isolated ECM is further micronized.

30. The method of claim 29, wherein the isolated ECM can be micronized to a particle size of about 1 to 1000 microns, 1 to 900 microns, 1 to 800 microns, 1 to 700 microns, 1 to 600 microns, 1 to 500 microns, 1 to 400 microns, 1 to 300 microns, 1 to 200 microns, 100 to 300 microns, 100 to 250 microns, 100 to 200 microns, 100 to 150 microns, or 100 to 125 microns.

31.-32. (canceled)

33. A method of treating and/or preventing a skin condition, wherein the method comprises administering the composition of claim 10 to a subject in need thereof.

34. The method of claim 33, wherein the skin condition comprises fine lines and/or wrinkles, aging, redness, abrasion, burns, cuts, infection, razor bumps, scars, uneven skin tone, pain, stretch marks, increasing skin elasticity and/or firmness, improve skin hydration, inflammation, and hyperpigmentation.

35. The method of claim 33, wherein preventing a skin condition comprises protecting the skin from damages comprising UV rays and/or environmental pollution.
